(19) United States
(12) Patent Application Publication
(10) Pub. No.: US 2026/0347059 A1
(43) Pub. Date: Nov. 5, 2026

(54) SEGMENTED LIQUID ADVANCING DEVICE

(71) Applicant: Dongguan Good Impression Industrial Co., Ltd., Dongguan (CN)

(72) Inventors: Wenben WU, Dongguan (CN); Zhibin LIANG, Dongguan (CN); Liping SHEN, Dongguan (CN)

(21) Appl. No.: 19/707,551

(22) Filed: Jun. 15, 2026

Related U.S. Application Data
(63) Continuation of application No. PCT/CN2025/082927, filed on Mar. 17, 2025.

(30) Foreign Application Priority Data
Mar. 20, 2024 (CN) 202420549167.2

(51) Int. Cl. B65D 41/16; A61M 5/315; B65D 51/24; B65D 83/76; B65D 83/764

(52) U.S. Cl. CPC B65D 41/16; A61M 5/31511; A61M 5/31526; A61M 5/31573; A61M 5/3158; B65D 51/24; B65D 83/764; B65D 83/768

(57) ABSTRACT

Disclosed is a segmented liquid advancing device, comprising: a chamber body and a cap body. The chamber body comprises an accommodating part and a metered advancing part. A medium is stored in the accommodating part. One end of the accommodating part is provided with a nozzle, and the metered advancing part is arranged at the other end of the accommodating part and used to quantitatively push out the medium within the accommodating part. The cap body is detachably connected to the end of the accommodating part provided with the nozzle.

### CROSS-REFERENCE TO RELATED APPLICATIONS

The present application is a continuation of PCT/CN2025/082927, filed Mar. 17, 2025 and claims priority of Chinese Patent Application No. 202420549167.2, filed on Mar. 20, 2024. The entire contents of International Patent Application No. PCT/CN2025/082927 and Chinese Patent Application No. 202420549167.2 are incorporated herein by reference.

### TECHNICAL FIELD

The present application relates to the technical field of liquid injection devices, and in particular, to a segmented liquid advancing device.

### BACKGROUND

A syringe is a common medical instrument, consisting of a barrel with a small hole at a front end thereof and a matching piston core rod, and is used for injecting a small amount of medium into areas inaccessible by other methods or extracting the medium from those areas.

For common handheld syringes, the operation is typically performed by manual pressing to achieve advancement, and the advancement amount is entirely controlled by an operator, resulting in poor dose accuracy. This is unsuitable for occasions with high requirements for dose accuracy, and this problem needs to be solved urgently.

### SUMMARY

In order to facilitate improvement in dose advancing accuracy, the present application provides a segmented liquid advancing device.

The segmented liquid advancing device according to the present application comprises: a chamber body, the chamber body comprising an accommodating part and a metered advancing part, wherein the accommodating part stores a medium, one end of the accommodating part has a nozzle, and the metered advancing part is arranged at the other end of the accommodating part and configured to quantitatively push out the medium within the accommodating part;
a cap body, the cap body being detachably connected to the end of the accommodating part having the nozzle.

By adopting the above technical solution, when the segmented liquid advancing device is not needed for use, the cap body is detachably connected to the end of the accommodating part having the nozzle to isolate and protect the medium within the accommodating part; when the segmented liquid advancing device is needed for use, an operator separates the cap body from the chamber body, and then quantitatively pushes out the medium within the accommodating part through the metered advancing part, thereby achieving metered use of doses, which facilitates improvement in dose advancing accuracy.

Preferably, the metered advancing part comprises a piston rod and a movable sleeve, the movable sleeve is movably sleeved on an end of the accommodating part away from the nozzle, the accommodating part is fixedly provided with a limiting step for preventing the movable sleeve from moving in a direction toward the nozzle, the movable sleeve is threadedly sleeved on the piston rod, a sealing sleeve is provided at an end of the piston rod facing the nozzle, the end of the piston rod provided with the sealing sleeve penetrates into the accommodating part, and a torque force of the threaded sleeving of the movable sleeve on the piston rod is less than a frictional force between the sealing sleeve and the accommodating part.

By adopting the above technical solution, before the segmented liquid advancing device is used, the movable sleeve abuts against the limiting step; when the operator rotates the movable sleeve, since the torque force of the threaded sleeving of the movable sleeve on the piston rod is less than the frictional force between the sealing sleeve and the accommodating part, the movable sleeve moves along a length direction of the piston rod, and the limiting step prevents the movable sleeve from moving in the direction toward the nozzle, so that the movable sleeve can only move in a direction away from the nozzle, at which point the movable sleeve has a space to move in the direction toward the nozzle; then the operator presses the movable sleeve in the direction toward the nozzle, and the movable sleeve drives the piston rod to push out the medium within the accommodating part until the movable sleeve abuts against the limiting step again, thereby achieving the quantitative push-out of the medium. The operator can control a movement distance of the movable sleeve by rotating the movable sleeve, thereby controlling a usage amount of the medium, which in turn facilitates improvement in dose advancing accuracy.

Preferably, the movable sleeve comprises a sleeve body and a connecting sleeve, the connecting sleeve is coaxially and fixedly connected inside the sleeve body, and the connecting sleeve is threadedly sleeved on the piston rod.

By adopting the above technical solution, the sleeve body is threadedly sleeved on the piston rod through the connecting sleeve, which can reduce a production diameter of the piston rod, and can reduce production costs while achieving the function of threaded sleeving connection.

Preferably, a limiting assembly is provided at an end of the accommodating part connected to the movable sleeve, the limiting assembly comprises a pushing member and an elastic member, the accommodating part is provided with an accommodating groove for accommodating the pushing member and the elastic member, the pushing member is proximate to the accommodating part, the elastic member is proximate to the metered advancing part, the pushing member moves with movement of the movable sleeve, one end of the elastic member abuts against the pushing member, and the other end of the elastic member abuts against a groove wall of the accommodating groove.

By adopting the above technical solution, when the movable sleeve moves in the direction away from the nozzle, the movable sleeve drives the pushing member to press against the elastic member, and the elastic member has a blocking force on the pushing member and the movable sleeve, which facilitates preventing the movable sleeve from completely disengaging from the accommodating part; at the same time, the elastic member has a tightening effect on the pushing member and the movable sleeve, thereby facilitating improvement in structural stability between the movable sleeve and the piston rod; in addition, when the operator presses the movable sleeve, a restoring force of the elastic member after being compressed has a facilitating effect on the operator's pressing operation of the movable sleeve, thereby facilitating the operator's pressing operation of the movable sleeve.

Preferably, the pushing member is configured as a pushing snap ring, an inner side of the sleeve body is provided with a snap groove, an inner side of the pushing snap ring is accommodated in the accommodating groove, and an outer side of the pushing snap ring is snap-fitted in the snap groove.

By adopting the above technical solution, the pushing snap ring is snap-fitted in the snap groove, so that the pushing snap ring can move with movement of the sleeve body, and simultaneously, the elastic member is pressed with the movement of the pushing snap ring.

Preferably, the elastic member is configured as a spring, and when a side of the pushing member facing away from the spring abuts against the groove wall of the accommodating groove, the spring is in a natural state.

By adopting the above technical solution, when the side of the pushing member facing away from the spring abuts against the groove wall of the accommodating groove, the spring is in the natural state; when the sleeve body drives the pushing snap ring to move in the direction away from the nozzle, the pushing snap ring presses the spring, so that the spring is in a compressed state, and a restoring force of the spring has a blocking effect on the movable sleeve, thereby facilitating preventing the movable sleeve from completely disengaging from the accommodating part; moreover, the restoring force of the spring tightens the movable sleeve through the pushing snap ring, thereby facilitating improvement in structural stability between the movable sleeve and the piston rod; in addition, a direction of the restoring force of the spring is the same as a direction of the operator's pressing force on the movable sleeve, which has a facilitating effect on the operator's pressing of the movable sleeve.

Preferably, a snap block is fixedly provided on an outer side wall of the chamber body, the cap body is sleeved on the accommodating part of the chamber body, and an inner side wall of the cap body is provided with a snap groove snap-fitted with the snap block.

By adopting the above technical solution, the cap body achieves detachable connection with the chamber body through snap-fitting between the snap block and the snap groove, and the structure is simple and convenient.

Preferably, the accommodating part is configured as a transparent accommodating chamber, and the cap body is provided with a window for observing the accommodating part.

By adopting the above technical solution, the operator can observe the medium within the accommodating part through the window, thereby facilitating the operator's use of the segmented liquid advancing device.

Preferably, a spring tab is provided at a position on the chamber body snap-fitted with the cap body, the spring tab protrudes in a direction toward the nozzle, the snap groove comprises a straight groove part and a hook groove part communicated with each other, the hook groove part and the straight groove part are arranged adjacent to each other in a left-right direction, and when the chamber body needs to be connected to the cap body, the snap block is aligned with the straight groove part.

By adopting the above technical solution, when the cap body needs to be connected to the chamber body, a user aligns the snap block with the straight groove part and pushes, so that the snap block is pushed into the straight groove part; at this time, the snap block is located at one side of the hook groove part, and simultaneously, the spring tab is pressed; then the user rotates the chamber body, so that the snap block enters the hook groove part; after the user releases, a restoring force of the spring tab after being pressed pushes the snap block deeper into the hook groove part; the spring tab limits the snap block in an axial direction of the chamber body, and the hook groove part limits the snap block in a direction perpendicular to the axis of the chamber body; in the absence of external force, the chamber body and the cap body are difficult to disengage, which facilitates improvement in stability of the connection between the chamber body and the cap body.

Preferably, a serpentine groove is provided at the position on the chamber body snap-fitted with the cap body, and the serpentine groove is obliquely arranged to form the spring tab.

By adopting the above technical solution, the provision of the serpentine groove facilitates improvement in elasticity of the spring tab, and the provision of the serpentine groove, while improving the elasticity of the spring tab, facilitates reduction of the weight of the segmented advancing device, thereby facilitating improvement in portability.

In summary, the present application includes at least one of the following beneficial technical effects:
1. By providing the piston rod and the movable sleeve, before the segmented liquid advancing device is used, the movable sleeve abuts against the limiting step; when the operator rotates the movable sleeve, since the torque force of the threaded sleeving of the movable sleeve on the piston rod is less than the frictional force between the sealing sleeve and the accommodating part, the movable sleeve moves along the length direction of the piston rod, and the limiting step prevents the movable sleeve from moving in the direction toward the nozzle, so that the movable sleeve can only move in the direction away from the nozzle, at which point the movable sleeve has a space to move in the direction toward the nozzle; then the operator presses the movable sleeve in the direction toward the nozzle, and the movable sleeve drives the piston rod to push out the medium within the accommodating part until the movable sleeve abuts against the limiting step again, thereby achieving the quantitative push-out of the medium. The operator can control the movement distance of the movable sleeve by rotating the movable sleeve, thereby controlling the usage amount of the medium, which in turn facilitates improvement in dose advancing accuracy.
2. By providing the pushing member and the elastic member, when the movable sleeve moves in the direction away from the nozzle, the movable sleeve drives the pushing member to press against the elastic member, and the elastic member has a blocking force on the pushing member and the movable sleeve, which facilitates preventing the movable sleeve from completely disengaging from the accommodating part; at the same time, the elastic member has a tightening effect on the pushing member and the movable sleeve, thereby facilitating improvement in structural stability between the movable sleeve and the piston rod; in addition, when the operator presses the movable sleeve, the restoring force of the elastic member after being compressed has a facilitating effect on the operator's pressing operation of the movable sleeve, thereby facilitating the operator's pressing operation of the movable sleeve.
3. By providing the pushing snap ring, the pushing snap ring is snap-fitted in the snap groove, so that the pushing snap ring can move with the movement of the sleeve body, and simultaneously, the elastic member is pressed with the movement of the pushing snap ring.

### BRIEF DESCRIPTION OF THE DRAWINGS

FIG. 1 is a schematic structural view of a segmented liquid advancing device according to Embodiment 1 of the present application when a chamber body and a cap body are separated.

FIG. 2 is a vertical cross-sectional view of the chamber body in Embodiment 1 of the present application.

FIG. 3 is a vertical cross-sectional view of a metered advancing part in Embodiment 1 of the present application.

FIG. 4 is an enlarged view of part A in FIG. 3.

FIG. 5 is a schematic structural view of a segmented liquid advancing device according to Embodiment 2 of the present application when a chamber body and a cap body are separated.

FIG. 6 is an enlarged view of part B in FIG. 5.

### LIST OF REFERENCE SIGNS

1. chamber body; 11. accommodating part; 12. metered advancing part; 121. piston rod; 122. movable sleeve; 1221. sleeve body; 1222. connecting sleeve; 2. nozzle; 3. cap body; 4. limiting step; 5. sealing sleeve; 6. limiting assembly; 61. pushing snap ring; 62. spring; 7. accommodating groove; 8. snap groove; 9. snap block; 10. snap groove; 101. straight groove part; 102. hook groove part; 13. window; 14. spring tab; 15. serpentine groove

### DETAILED DESCRIPTION

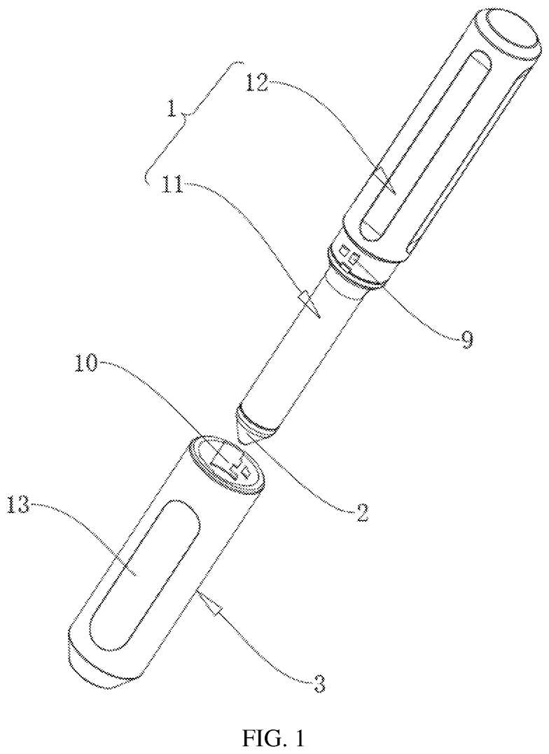

The present application is further described in detail below in conjunction with FIGS. 1-6.

### Embodiment 1

Embodiment 1 of the present application provides a segmented liquid advancing device. Referring to FIG. 1, the device comprises a chamber body 1 and a cap body 3, and the chamber body 1 and the cap body 3 are detachably connected, so as to facilitate storage and use of the segmented liquid advancing device. Specifically, a snap block 9 is integrally formed on an outer side wall of the chamber body 1; meanwhile, the cap body 3 is sleeved on the chamber body 1, and an inner side wall of the cap body 3 is provided with a snap groove 10 snap-fitted with the snap block 9. The cap body 3 and the chamber body 1 achieve detachable connection through snap-fitting between the snap block 9 and the snap groove 10, which is simple and convenient.

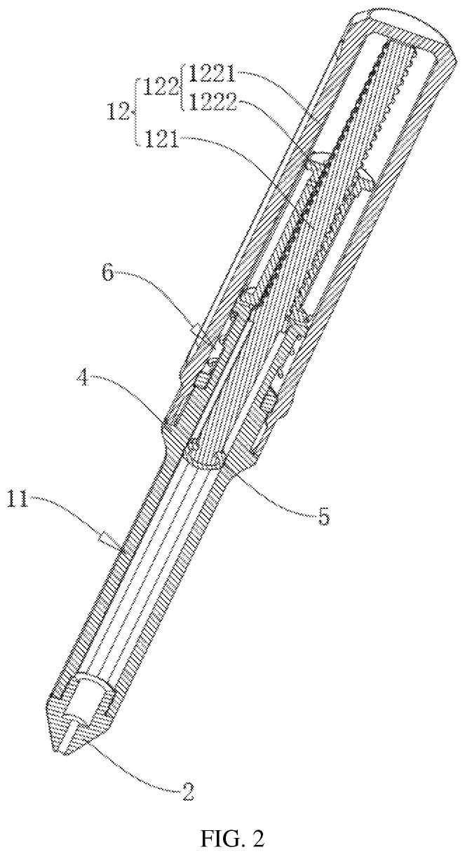

Referring to FIG. 1 and FIG. 2, the chamber body 1 comprises an accommodating part 11 and a metered advancing part 12. The accommodating part 11 stores a medium therein, and one end of the accommodating part 11 has a nozzle 2 for the medium to flow out. The cap body 3 is sleeved on the chamber body 1 from the end of the accommodating part 11 having the nozzle 2, so as to isolate and protect the medium within the accommodating part 11. The metered advancing part 12 is arranged at the other end of the accommodating part 11 and configured to quantitatively push out the medium within the accommodating part 11 from the nozzle 2, thereby achieving metered use of doses without requiring the operator to manually control the advancement dose, which can improve dose advancing accuracy. It should be noted that the accommodating part 11 is configured as a transparent accommodating chamber; meanwhile, a side wall of the cap body 3 is fixedly provided with a window 13 along a length direction thereof for observing the accommodating part 11. The operator can observe the medium within the accommodating part 11 through the window 13, thereby facilitating the operator's use of the segmented liquid advancing device.

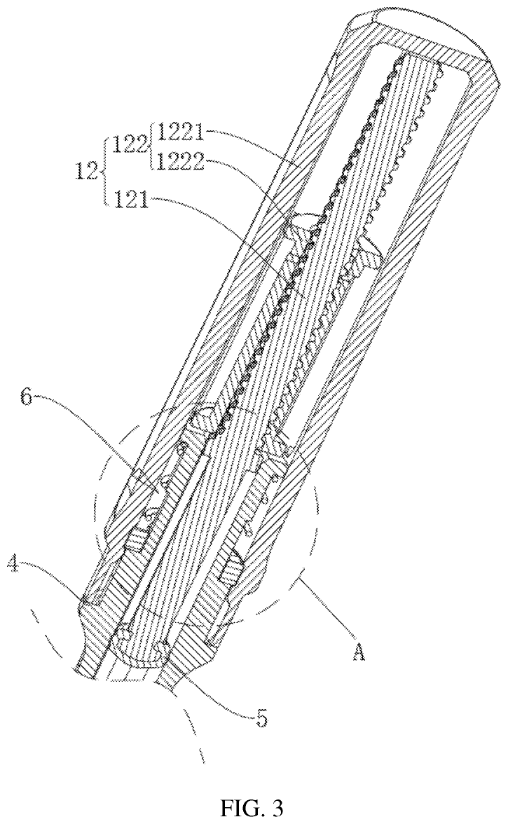

Referring to FIG. 2 and FIG. 3, the metered advancing part 12 comprises a piston rod 121 and a movable sleeve 122. The movable sleeve 122 is movably sleeved on an end of the accommodating part 11 away from the nozzle 2; meanwhile, the accommodating part 11 is fixedly provided with a limiting step 4 for preventing the movable sleeve 122 from moving in a direction toward the nozzle 2, and the limiting step 4 is fixedly and circumferentially arranged around a peripheral side of the accommodating part 11. The movable sleeve 122 is threadedly sleeved on the piston rod 121, a sealing sleeve 5 is provided at an end of the piston rod 121 facing the nozzle 2, and the end of the piston rod 121 provided with the sealing sleeve 5 penetrates into the accommodating part 11. In the present embodiment, the sealing sleeve 5 is made of a soft silicone material, so as to improve sealing performance of the accommodating part 11 and the frictional force between the piston rod 121 and the accommodating part 11. It should be noted that a torque force of the threaded sleeving of the movable sleeve 122 on the piston rod 121 is less than a frictional force between the sealing sleeve 5 and the accommodating part 11.

Before the segmented liquid advancing device is used, one end of the movable sleeve 122 abuts against the limiting step 4. When the operator rotates the movable sleeve 122, since the torque force of the threaded sleeving of the movable sleeve 122 on the piston rod 121 is less than the frictional force between the sealing sleeve 5 and the accommodating part 11, the movable sleeve 122 moves along the length direction of the piston rod 121, and the limiting step 4 prevents the movable sleeve 122 from moving in the direction toward the nozzle 2, so that the movable sleeve 122 can only move in the direction away from the nozzle 2. At this point, the movable sleeve 122 has a space to move in the direction toward the nozzle 2. Then the operator presses the movable sleeve 122 in the direction toward the nozzle 2, and the movable sleeve 122 drives the piston rod 121 to push out the medium within the accommodating part 11 until the movable sleeve 122 abuts against the limiting step 4 again, thereby achieving the quantitative push-out of the medium. The operator can control the movement distance of the movable sleeve 122 by rotating the movable sleeve 122, thereby controlling the usage amount of the medium, which in turn facilitates improvement in dose advancing accuracy.

Referring to FIG. 2 and FIG. 3, the movable sleeve 122 comprises a sleeve body 1221 and a connecting sleeve 1222. The connecting sleeve 1222 is coaxially and fixedly connected inside the sleeve body 1221, and the length of the connecting sleeve 1222 is less than the length of the sleeve body 1221. The connecting sleeve 1222 is threadedly sleeved on the piston rod 121. The sleeve body 1221 is threadedly sleeved on the piston rod 121 through the connecting sleeve 1222, and while satisfying the function of the movable sleeve 122 being threadedly sleeved on the piston rod 121, the production diameter of the piston rod 121 can be reduced, or the thickness of the sleeve body 1221 can be reduced, thereby facilitating reduction of production costs and making the segmented liquid advancing device more lightweight.

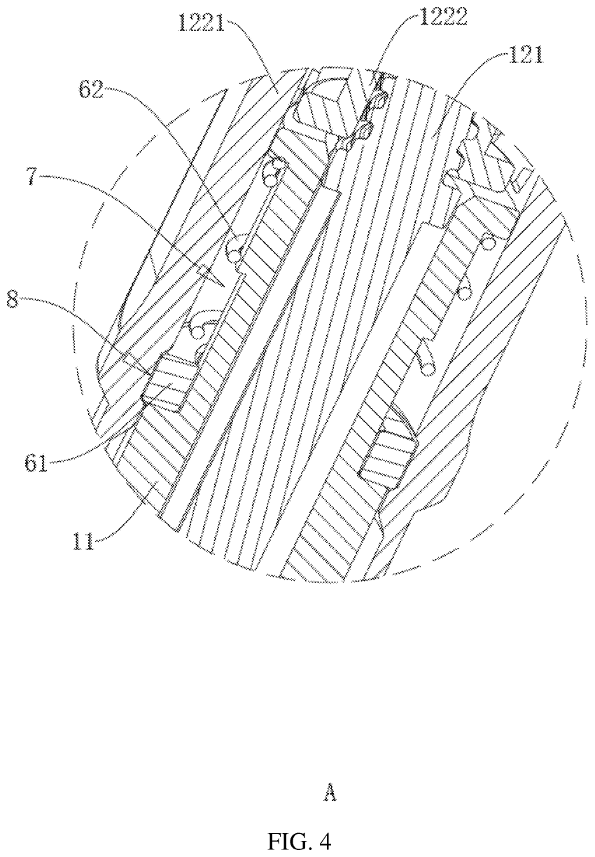

Referring to FIG. 3 and FIG. 4, a limiting assembly 6 for limiting the movement of the sleeve body 1221 is provided at the end of the accommodating part 11 movably sleeved with the sleeve body 1221, so as to prevent the sleeve body 1221 from completely disengaging from the accommodating part 11. Specifically, the limiting assembly 6 comprises a pushing member and an elastic member. An outer side wall of the accommodating part 11 is circumferentially provided with an accommodating groove 7 for accommodating the pushing member and the elastic member. It should be noted that the pushing member is located at an end within the accommodating groove 7 proximate to the accommodating part 11, and the elastic member is located at an end within the accommodating groove 7 proximate to the metered advancing part 12. The pushing member moves with the movement of the movable sleeve 122, one end of the elastic member abuts against the pushing member, and the other end of the elastic member abuts against a groove wall of the accommodating groove 7.

When the movable sleeve 122 rotates and moves in the direction away from the nozzle 2, the sleeve body 1221 drives the pushing member to synchronously move in the direction away from the nozzle 2, and the pushing member presses the elastic member during the movement. A restoring force generated after the elastic member is pressed blocks the movement of the pushing member, making it difficult for the sleeve body 1221 to completely disengage from the accommodating part 11. In the present embodiment, the pushing member is configured as a pushing snap ring 61, and the elastic member is configured as a spring 62. An inner side of the sleeve body 1221 is circumferentially provided with a snap groove 8. An inner side of the pushing snap ring 61 is circumferentially accommodated in the accommodating groove 7, and an outer side of the pushing snap ring 61 is circumferentially snap-fitted in the snap groove 8, so that the movement of the sleeve body 1221 can synchronously drive the pushing snap ring 61 to move. Meanwhile, when a side of the pushing snap ring 61 facing away from the spring 62 abuts against the groove wall of the accommodating groove 7, the spring 62 is in a natural state. It should be noted that the provision of the spring 62 and the pushing snap ring 61 can not only prevent the sleeve body 1221 from completely disengaging from the accommodating part 11, but the elastic force of the spring 62 can also enhance structural stability, which facilitates preventing various connection structures within the chamber body 1 from shaking due to assembly gaps, thereby facilitating further improvement in dose advancing accuracy. In addition, when the operator presses the movable sleeve 122, the restoring force of the spring 62 after being compressed has a facilitating effect on the operator's pressing operation of the movable sleeve 122, thereby facilitating the operator's pressing operation of the movable sleeve 122.

The implementation principle of the segmented liquid advancing device according to Embodiment 1 of the present application is as follows: before the segmented liquid advancing device is used, the sleeve body 1221 is in a state of abutting against the limiting step 4. When the segmented liquid advancing device needs to be used for metered use of the medium, the operator rotates the movable sleeve 122. Since the torque force of the threaded sleeving of the movable sleeve 122 on the piston rod 121 is less than the frictional force between the sealing sleeve 5 and the accommodating part 11, the piston rod 121 remains stationary, while the movable sleeve 122 moves along the length direction of the piston rod 121. The limiting step 4 prevents the movable sleeve 122 from moving in the direction toward the nozzle 2, so the movable sleeve 122 can only move in the direction away from the nozzle 2. At this point, the movable sleeve 122 has a space to move in the direction toward the nozzle 2. Then the operator presses the movable sleeve 122 in the direction toward the nozzle 2, and the movable sleeve 122 drives the piston rod 121 to push out the medium within the accommodating part 11 until the movable sleeve 122 abuts against the limiting step 4 again, thereby achieving the quantitative push-out of the medium. The operator can control the movement distance of the movable sleeve 122 by rotating the movable sleeve 122, thereby controlling the usage amount of the medium, which in turn facilitates improvement in dose advancing accuracy.

### Embodiment 2

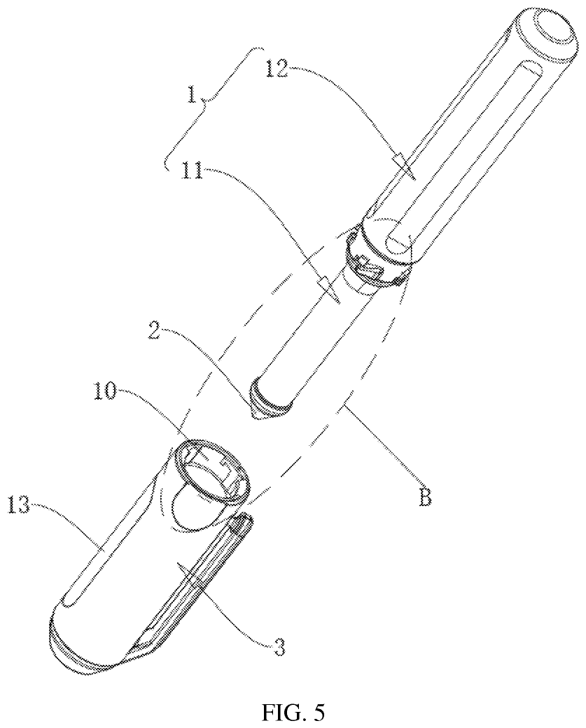

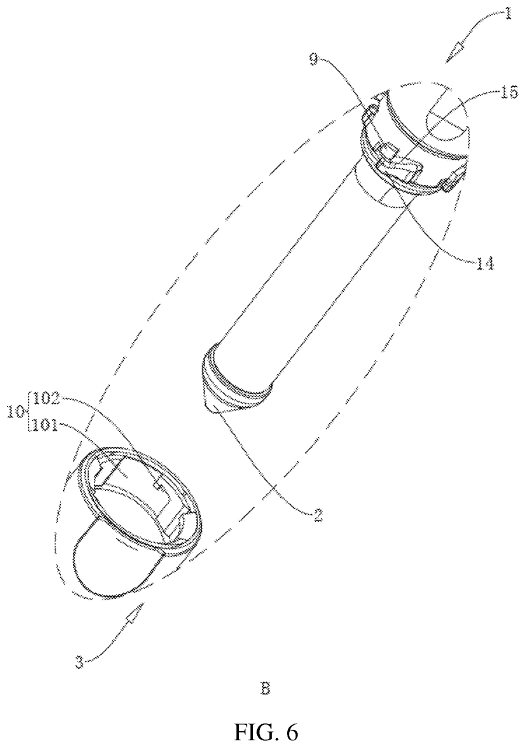

Referring to FIG. 5 and FIG. 6, the difference from Embodiment 1 lies in that a spring tab 14 is provided at a position on the chamber body 1 snap-fitted with the cap body 3, the spring tab 14 protrudes in a direction toward the nozzle 2, the snap groove 10 comprises a straight groove part 101 and a hook groove part 102 communicated with each other, the hook groove part 102 and the straight groove part 101 are arranged adjacent to each other in a left-right direction, and when the chamber body 1 needs to be connected to the cap body 3, the snap block 9 is aligned with the straight groove part 101. It should be noted that a serpentine groove 15 is provided at the position on the chamber body 1 snap-fitted with the cap body 3, and the serpentine groove 15 is obliquely arranged to form the spring tab 14. The provision of the serpentine groove 15 facilitates improvement in elasticity of the spring tab 14, and the provision of the serpentine groove 15, while improving the elasticity of the spring tab 14, facilitates reduction of the weight of the segmented advancing device, thereby facilitating improvement in portability.

When the cap body 3 needs to be connected to the chamber body 1, a user aligns the snap block 9 with the straight groove part 101 and pushes, so that the snap block 9 is pushed into the straight groove part 101. At this time, the snap block 9 is located at one side of the hook groove part 102, and simultaneously, the spring tab 14 is pressed. Then the user rotates the chamber body 1, so that the snap block 9 enters the hook groove part 102. After the user releases, a restoring force of the spring tab 14 after being pressed pushes the snap block 9 deeper into the hook groove part 102. The spring tab 14 limits the snap block 9 in an axial direction of the chamber body 1, and the hook groove part 102 limits the snap block 9 in a direction perpendicular to the axis of the chamber body 1. In the absence of external force, the chamber body 1 and the cap body 3 are difficult to disengage, which facilitates improvement in stability of the connection between the chamber body 1 and the cap body 3.

The implementation principle of the segmented liquid advancing device of Embodiment 2 of the present application is substantially the same as that of Embodiment 1, and will not be repeated herein.

The above are all preferred embodiments of the present application, and are not intended to limit the protection scope of the present application thereby. Therefore, any equivalent changes made according to the structure, shape, and principle of the present application shall all fall within the protection scope of the present application.

1. A segmented liquid advancing device, comprising:
a chamber body, comprising an accommodating part and a metered advancing part, wherein the accommodating part stores a medium, a first end of the accommodating part is provided with a nozzle, and the metered advancing part is arranged at a second end of the accommodating part and configured to quantitatively push out the medium within the accommodating part; and
a cap body, detachably connected to the first end of the accommodating part provided with the nozzle.

2. The segmented liquid advancing device according to claim 1, wherein the metered advancing part comprises a piston rod and a movable sleeve, the movable sleeve is movably sleeved on the second end of the accommodating part away from the nozzle, the accommodating part is fixedly provided with a limiting step for preventing the movable sleeve from moving in a direction toward the nozzle, the movable sleeve is threadedly sleeved on the piston rod, a sealing sleeve is provided at an end of the piston rod facing the nozzle, the end of the piston rod provided with the sealing sleeve penetrates into the accommodating part, and a torque force of the threaded sleeving of the movable sleeve on the piston rod is less than a frictional force between the sealing sleeve and the accommodating part.

3. The segmented liquid advancing device according to claim 2, wherein the movable sleeve comprises a sleeve body and a connecting sleeve, the connecting sleeve is coaxially and fixedly connected inside the sleeve body, and the connecting sleeve is threadedly sleeved on the piston rod.

4. The segmented liquid advancing device according to claim 3, wherein a limiting assembly is provided at the second end of the accommodating part connected to the movable sleeve, the limiting assembly comprises a pushing member and an elastic member, the accommodating part is provided with an accommodating groove for accommodating the pushing member and the elastic member, the pushing member is proximate to the accommodating part, the elastic member is proximate to the metered advancing part, the pushing member moves with movement of the movable sleeve, a first end of the elastic member abuts against the pushing member, and a second end of the elastic member abuts against a groove wall of the accommodating groove.

5. The segmented liquid advancing device according to claim 4, wherein the pushing member is configured as a pushing snap ring, an inner side of the sleeve body is provided with a snap groove, an inner side of the pushing snap ring is accommodated in the accommodating groove, and an outer side of the pushing snap ring is snap-fitted in the snap groove.

6. The segmented liquid advancing device according to claim 4, wherein the elastic member is configured as a spring, and when a side of the pushing member facing away from the spring abuts against the groove wall of the accommodating groove, the spring is in a natural state.

7. The segmented liquid advancing device according to claim 1, wherein a snap block is fixedly provided on an outer side wall of the chamber body, the cap body is sleeved on the accommodating part of the chamber body, and an inner side wall of the cap body is provided with a snap groove snap-fitted with the snap block.

8. The segmented liquid advancing device according to claim 1, wherein the accommodating part is configured as a transparent accommodating chamber, and the cap body is provided with a window for observing the accommodating part.

9. The segmented liquid advancing device according to claim 7, wherein a spring tab is provided at a position on the chamber body snap-fitted with the cap body, the spring tab protrudes in the direction toward the nozzle, the snap groove comprises a straight groove part and a hook groove part in communication with each other, the hook groove part and the straight groove part are arranged adjacent to each other in a left-right direction, and when the chamber body needs to be connected to the cap body, the snap block is aligned with the straight groove part.

10. The segmented liquid advancing device according to claim 9, wherein a serpentine groove is provided at the position on the chamber body snap-fitted with the cap body, and the serpentine groove is obliquely arranged to form the spring tab.
